(19) United States
(12) Patent Application Publication
(10) Pub. No.: US 2023/0347065 A1
(43) Pub. Date: Nov. 2, 2023

(54) ELECTRONIC SYSTEM FOR A DRUG DELIVERY DEVICE, DRUG DELIVERY DEVICE, AND ASSOCIATED METHOD

(71) Applicant: Sanofi, PARIS (FR)

(72) Inventors: Paul Richard Draper, Evesham, Worcestershire (GB); Aidan Michael O`Hare, Warwick, Warwickshire (GB); William Geoffrey Arthur Marsh, Warwick, Warwickshire (GB); Richard Paul Gledhill, Warwick, Warwickshire (GB); Anthony Paul Morris, Coventry, West Midlands (GB); Daniel Paul Jenkins, Warwick, Warwickshire (GB); Tom Alexander Earwaker, Warwick, Warwickshire (GB); Oliver Charles Gazeley, Basel (CH); Adam Moyo Harvey-Cook, Billericay, Essex (GB); Stefan Alt, Frankfurt am Main (DE)

(21) Appl. No.: 18/016,941

(22) PCT Filed: Jul. 21, 2021
(86) PCT No.: PCT/EP2021/070315

§ 371 Date: Jan. 19, 2023

(30) Foreign Application Priority Data
Jul. 23, 2020 (EP) 20315356.4

(51) Int. Cl. A61M 5/315

(52) U.S. Cl. CPC A61M 5/31546; A61M 2205/8212; A61M 2205/505

(57) ABSTRACT

An electronic system comprising: at least one user interface member configured to be manipulated by a user for performing a dose setting operation of the drug delivery device and/or for performing a dose delivery operation for delivering a set dose, an electronic control unit configured to control operation of the electronic system, an electrical signaling unit configured to provide at least one electrical signal when the user interface member is manipulated, and a manipulation evaluation unit configured to evaluate the at least one electrical signal of the signaling unit to determine whether the manipulation of the user interface member indicated by the at least one signal of the signaling unit qualifies as an activation operation. The electronic control unit is configured to switch the electronic system into a higher power consumption when the manipulation evaluation unit has confirmed that the manipulation qualifies as activation operation.

### CROSS-REFERENCE TO RELATED APPLICATIONS

The present application is the national stage entry of International Patent Application No. PCT/EP2021/070315, filed on Jul. 21, 2021, and claims priority to Application No. EP 20315356.4, filed on Jul. 23, 2020, the disclosures of which are incorporated herein by reference.

### TECHNICAL FIELD

The present disclosure relates to an electronic system for a drug delivery device. The present disclosure further relates to a drug delivery device, which preferably comprises the electronic system. The present disclosure further relates to a method for preparing an electronic system for an operation, e.g., an operation of the drug delivery device such as a dose setting operation or a dose delivery operation.

### BACKGROUND

Drug delivery devices using electronics are becoming increasingly popular in the pharmaceutical industry as well as for users or patients. However, especially if the device is designed to be self-contained, that is to say without a connector for a connection to an external electrical power source which is necessary to provide electrical power for the operation of the device, the management of the resources of a power supply integrated into the device is particularly important.

### SUMMARY

The present disclosure provides improvements for drug delivery devices comprising an electronic system or electronic systems for drug delivery devices.

One aspect of the present disclosure relates to an electronic system for a drug delivery device. Another aspect of the present disclosure relates to a drug delivery device, especially one comprising the electronic system. Yet another aspect relates to a method of preparing an electronic system for a drug delivery device or a drug delivery device with an electronic system, e.g., for a dose delivery operation. Still another aspect relates to a computer program product, which contains machine-readable instructions, which, when loaded and executed on a processor are suitable to execute or control execution of at least one of, an arbitrarily selected plurality of or all of the steps of the method. Accordingly, the features, which are disclosed in relation to the drug delivery device or units thereof or therefore, do also apply for the electronic system, the method and the computer program product and vice versa.

In one embodiment, the electronic system comprises at least one user interface member. The user interface member may be arranged or configured to be manipulated, e.g., touched and/or moved, by the user of the drug delivery device. The user interface member may be provided for performing a dose setting operation to set a dose of drug to be delivered by the drug delivery device and/or for performing a dose delivery operation for delivering a set dose, preferably the one which has been previously set during the dose setting operation. The respective operation may be performed by the user of the drug delivery device, e.g., a patient. The respective operation may require continuous contact of the user to the user interface member throughout the entire operation.

In one embodiment, the user interface member has an exterior operation surface arranged and configured to be touched by a user, e.g., by the user of the drug delivery device. The exterior operation surface may be arranged and configured to be touched during operation of the system or the device, such as the dose setting operation and/or the dose delivery operation, e.g., for initiating and/or for performing the operation. Thus, the exterior operation surface may be or may comprise a setting surface for the setting operation and/or a delivery surface for the delivery operation. The setting surface and the delivery surface may face in different directions. The setting surface may face in the radial or lateral direction. The delivery surface may face in an axial, e.g., in a proximal, direction. Alternatively or additionally to the dose setting operation and/or the dose delivery operation, the user interface member may be configured to be manipulated for an activation operation.

In one embodiment, the electronic system comprises an electronic control unit. The electronic control unit may be configured to control an operation of the electronic system. The electronic control unit may be or may comprise an electronic processor, such as a microcontroller or an ASIC, for example. The electronic system may have a first state and a second state, e.g. when it is operative. The electronic system may have an increased electrical power consumption in the second state as compared to the first state. In the first state, one or more electrical or electronic units of the electronic system may be in a sleep mode or be powered off such that they have no significant power consumption or no power consumption. For example, in the second state a motion sensing unit may be active, i.e. it can be operated, where this unit is not active, i.e. it cannot be operated, in the first state. The motion sensing unit will be described in more detail below. Alternatively or additionally, a communication unit may be inactive in the first state and active in the second state. The communication unit will be described in more detail below.

In one embodiment, the electronic system comprises an electrical signaling unit. The unit may be configured to provide at least one electrical signal when the user interface is manipulated. For example, the at least one signal may be initiated when the user commences movement of the user interface member, for example relative to a housing. The housing may be a housing of the drug delivery device or of a drug delivery device unit to which the electronic system should be connected. The respective electrical signal may require movement for being generated. Alternatively, the electrical signal may be generated when the user touches or is in proximity to an exterior surface of the user interface member such as the setting surface or delivery surface. The electrical signaling unit may be configured to provide a signal or a signal sequence or pattern, which allows to determine, whether the manipulation which the user performs with the user interface member is an activation operation.

In one embodiment, the electronic control unit is configured to switch the electronic system from the first state into the second state, e.g., by issuing an according command or signal.

In one embodiment, the electronic system comprises a manipulation evaluation unit. The manipulation evaluation unit may be operatively connected to the electrical signaling unit. The manipulation evaluation unit may be configured to evaluate the at least one electrical signal provided by the signaling unit, preferably in order to determine whether the manipulation of the user interface member indicated by the at least one signal of the signaling unit qualifies as an activation operation. The at least one signal may consist of just one signal and the manipulation evaluation unit may evaluate the signal or at least a portion thereof or may consist of a sequence of different or successive signals. The manipulation evaluation unit may determine, whether the signal or the sequence of signals meets one or more predetermined criteria. If the criteria are met, the manipulation is assessed as being an activation operation. If the activation operation has been confirmed or affirmed by the manipulation evaluation, the operation of the system can be influenced in response to this activation operation. The manipulation may be qualified as an activation operation, for example only if a characteristic portion of the signal is detected by the evaluation unit and/or if a predetermined signal shape and/or signal sequence is detected by the evaluation unit.

In one embodiment, the manipulation of the user interface member for the activation operation is different from the manipulation of the user interface member for the dose setting operation and/or the dose delivery operation. That is to say, in order to qualify as an activation operation, the manipulation of the user interface member which is required for the activation operation may have to be different from at least one of or both of the dose setting operation and the dose delivery operation, preferably also from a dose correction operation which reduces the size of a set dose. In this way, a manipulation which is unique for the operation of the device may be used to indicate a manipulation which qualifies as activation operation. Hence, a user may decide on whether to perform the activation operation or the dose setting operation or the dose delivery operation.

In one embodiment, the manipulation for the activation operation is unique. That is to say only one defined manipulation may qualify as activation operation.

In one embodiment, the electronic control unit is configured to switch the electronic system into the second state of higher power consumption when the manipulation evaluation unit has confirmed or affirmed that the manipulation qualifies as activation operation. That is to say, the electronic control unit may be configured such that the system is only switched to the state of higher power consumption, e.g. with activated communication unit and/or activated motion sensing unit, if a manipulation has been detected which qualifies as activation operation. In order to qualify as activation operation the manipulation may have to meet a variety of criteria. If one of the criteria is not met by the manipulation, the manipulation may not be classified or may not qualify as an activation operation. In this way, a user of the electronic system or the device can decide whether the electronic functionality which may be available in the second state is needed currently or not. If the functionality is needed, the user may decide to perform the activation operation before conducting the operation in order to activate the electronic functionality, e.g., a delivered dose recording functionality and/or dose data transmission functionality of the system. One or more electronic functionalities may be available in the first state, however, with a lower power consumption than in the second state. For example, in the first state, the electrical signaling unit should be operational, to generate the electrical signal. The signaling unit may comprise an electronic component. Alternatively, in the first state no electronic functionality may be available, expediently aside from the one of the signaling unit.

In one embodiment, when the manipulation evaluation unit has qualified a manipulation as an activation operation, the manipulation evaluation unit may issue a use signal or activation prompt signal. The signal may be indicative that a completed activation operation has been confirmed by the manipulation evaluation unit. The electronic control unit may be configured to switch the electronic system to the second state of higher power consumption in response to the signal. In the second state, as opposed to the first state, the motion sensing unit and/or the communication unit may be operational. Preferably, at least the motion sensing unit is operational in the second state.

In one embodiment, in the second state, the electronic system is configured to gather information or data related to the size of the currently dispensed or delivered dose during the delivery operation, e.g., via the motion sensing unit. Consequently, the motion sensing unit may be configured to contribute to retrieve dose data on the dose delivered in the delivery operation, e.g. the currently delivered dose during the dose delivery operation.

In one embodiment, in the second state, the electronic system is configured to store dose data in a dose memory or memory unit of the electronic system. The memory may be transitory or non-transitory. The dose data is expediently derived using measurements or signals of the motion sensing unit.

In one embodiment, in the second state, the electronic system is configured to transmit dose data, e.g. dose data retrieved from the memory, by means of the communication unit to another device or system such as a computing device, e.g., a mobile phone or a portable or non-portable computing unit.

In one embodiment, the manipulation evaluation unit operates purely software based. That is to say, there is no need to provide additional hardware for evaluating whether a manipulation of the user interface member, which is expediently different from the dose setting operation and the dose delivery operation, qualifies as an activation operation.

In one embodiment, the manipulation which qualifies as activation operation requires a sequence of movements, e.g., a sequence of different movements, of the user interface member. The movements may differ in direction, for example. The movement of the user interface member may be relative to a housing, e.g., of the electronic system, of the drug delivery device or a drug delivery device unit. In order for the manipulation to qualify as activation operation, the sequence of movements may have to be completed within a predetermined time. The predetermined time may be less than or equal to one of the following values: 15 seconds (s), 10 s, 8 s, 6 s, 5 s, 4 s, 3 s, 2 s. If movement(s), which would, merely by their nature or the associated signals generated by the signaling unit, qualify the manipulation as an activation operation are not completed within the predetermined time, the manipulation is expediently not qualified as an activation operation by the manipulation evaluation unit.

In one embodiment, the manipulation which qualifies as activation operation requires movements of the user interface member in different directions, preferably in opposite directions, e.g., in opposite axial directions. One of the movements in the different directions may be user-driven, e.g., the initial movement, whereas the other movement, e.g., the one in the opposite direction may be driven by a resilient member which may be biased during the user-driven movement.

In one embodiment, the manipulation which qualifies as activation operation requires a repetition of one sequence of movements of the user interface member. The sequence of movements of the user interface member, which may have to be repeated, may comprise movements in different directions, such as opposite axial directions. The movements in the different directions may be movements relative to the housing. In other words, one sequence of movements may have to be performed several times, e.g., twice, such that the manipulation is qualified as an activation operation by the manipulation evaluation unit, when the evaluation unit evaluates the signals generated by the signaling unit.

In one embodiment, during each of the movements of the sequence of different movements, one or more (characteristic) electrical signals or (characteristic) portions of one signal or (characteristic) signal pulses may be generated. This facilitates evaluation of the manipulation by the manipulation evaluation unit based on the at least one signal, e.g., based on portion(s), sequence and/or duty cycle of the signal(s).

In one embodiment, the electronic system or the drug delivery device comprises a housing. For conducting the dose setting operation and/or the dose delivery operation, the user interface member may be moved relative to the housing, e.g., rotationally and axially away from the housing for dose setting and axially displaced towards the housing for dose delivery.

In one embodiment, the user interface member is movable, e.g., axially, from a first position to a second position, for example relative to the housing, such as the housing of the system or the drug delivery device. The first position may be an initial position which the user interface member has relative to the housing, e.g. before a dose setting operation and/or a dose delivery operation is commenced. The second position may be a position which the user interface member assumes when a user force is applied to the user interface member, e.g., a distally directed force, and the user interface member is moved away from the first position. The first position and the second position may be axially offset. The movement from the first position to the second position may involve only axial movement. The manipulation which qualifies as activation operation may require at least one, e.g., only one, movement towards the first position, e.g., from the second position towards the first position. That is to say, the manipulation may require a movement from the first position to the second position and the subsequent movement from the second position to the first position.

In one embodiment, the electronic system or the drug delivery device comprises a dose setting and/or drive mechanism. The dose setting and/or drive mechanism may comprise a first member and a second member. The first member and/or the second member may be configured to move during the dose setting operation and/or the dose delivery operation relative to the housing of the electronic system or the drug delivery device. The first member may be a dose member or dial member of the dose setting and/or drive mechanism, which is moved to set a dose, e.g., a dial sleeve or a number sleeve. The second member may be a drive member, e.g., a member engaged with a piston rod of the dose setting and/or drive mechanism, or a device user interface member, such as a dose knob and/or injection button. The first member and/or the second member may be movably coupled to or retained in the housing. In the dose setting operation, the first member and/or the second member may be displaced axially relative to the housing, for example away from a proximal end of the housing. The distance by which the first member and/or the second member is displaced during the dose setting operation relative to the housing, e.g., axially, may be determined by the size of the set dose. In other words, the drug delivery device may be of the dial extension type, i.e., the device increases its length during the dose setting operation in an amount proportional to the size of the set dose.

In one embodiment, in the dose setting operation and/or in the dose delivery operation, the first member moves, e.g. rotates and/or moves axially, relative to the second member. For example, the first member may rotate relative to the second member during the dose delivery operation, e.g. only during the dose delivery operation. The first member and the second member may both move axially during the dose delivery operation. The first member may rotate relative to the second member and relative to the housing during the dose setting operation and/or the dose delivery operation. The second member may be rotationally locked or guided with respect to the housing during the dose delivery operation, e.g., by a delivery clutch. The first member and the second member may be rotationally locked relative to one another during the dose setting operation. Accordingly, the first member and the second member may rotate relative to the housing in the dose setting operation. During the dose setting operation the first member and the second member may be coupled to one another, e.g. via a coupling interface, e.g., a setting clutch. The coupling interface may rotationally lock the first member and the second member to one another during the dose setting operation. When the coupling interface is engaged, the first member and the second member may be rotationally locked with one another, such as by direct engagement of coupling interface features. The first member and the second member may comprise mating coupling interface features. The coupling interface may be released during the dose delivery operation, e.g., by axially displacing the second member relative to the first member. Hence, the second member may be rotationally locked relative to the housing during dose delivery, whereas the first member may rotate relative to the housing during dose delivery.

The coupling interface may be released when switching the dose setting and/or drive mechanism from a dose setting configuration into a dose delivery configuration. This may be achieved when the user interface member is moved from the first position to the second position. In the first position, the mechanism may be in the dose setting configuration. In the second position, the mechanism may be in the dose delivery configuration.

In one embodiment, the first member and the second member rotate relative to one another during only one of the dose setting operation and the dose delivery operation. One of the first member and the second member, e.g. the first member, may rotate relative to the housing during both operations. One of the first member and the second member, e.g., the second member, may rotate relative to the housing during only one of the operations, e.g., during dose setting or during dose delivery.

In one embodiment, the electronic system comprises at least one of, an arbitrarily selected plurality of, or all of the following units or components:
an electrical motion sensing unit. The motion sensing unit will be explained in more detail below.
a communication unit. The communication unit may be provided to establish the communication interface between the electronic system and another device such as an electronic device such as a portable device, e.g. a portable or non-portable computer, a mobile phone or a tablet. The communication unit may be a wireless unit, e.g. an RF communication unit, such as a Bluetooth unit. The communication unit may be provided to transmit dose data from the electronic system to the other device, e.g. information on the amount of drug delivered by the device in a delivery operation.
a memory unit. The memory unit may be provided to store executable program code and/or data on dose information which has been calculated by the electronic system, preferably dose data on the delivered dose or doses. The dose data may be determined via the motion sensing unit. From the memory unit, the data may be retrievable for transmission to another device, e.g. via the communication unit.

In one embodiment, the motion sensing unit is configured to generate one or more electrical motion signals. The motion signal(s) may be suitable to quantify the relative movement between the first member and the second member, e.g. during the dose setting operation or the dose delivery operation, e.g. to obtain dose data, such as the size of the delivered dose. The first member and/or the second member may be members of the electronic system and/or the drug delivery device, e.g. the dose setting and/or drive mechanism as discussed further above. The relative movement may be relative rotational movement. For example, the first member may rotate relative to the second member during dose delivery.

In one embodiment, the electronic system is configured such that the motion sensing unit is switched from the first state into the second state, e.g. by the electronic control unit and/or in response to the use or activation prompt signal. In the first state, the motion sensing unit may be not operative to sense movement of the first member relative to the second member. In the second state, the motion sensing unit may be operative. In the second state, the motion sensing unit may have a power consumption which is greater than in the first state. The increase in power consumption of the motion sensing unit may contribute to or define the increased power consumption of the electronic system in the second state.

In one embodiment, the motion sensing unit is configured to operate during the dose delivery operation, preferably only during the dose delivery operation. The motion sensing unit may be configured to monitor the dose delivery operation, e.g. the rotation of the first member relative to the second member. Thus, from the motion signals, positional information on the relative position between the first member and the second member can be gathered. Alternatively or additionally, it is also possible to gather positional information between two members in the dose setting operation. However, in order to calculate dose information or data on the dose delivery during the dose delivery operation, it is advantageous to monitor the movements during the dose delivery operation by the motion sensing unit.

In one embodiment, the electronic control unit or the electronic system is configured to calculate dose information or data utilizing the motion signals generated by the motion sensing unit. As noted previously, the dose information preferably is information on the size of the dose which is delivered in the dose delivery operation.

In one embodiment, the motion sensing unit comprises one or more sensors and/or one or more emitters, e.g. one or more optoelectronic radiation sensors or detectors and/or one or more optoelectronic radiation emitters. The sensors may be configured to generate motion signal(s) in response to movement of the first member relative to the second member. The emitters may excite the sensor signals.

In one embodiment, during the dose setting operation, the dose may be set, e.g. between a minimum settable dose and a maximum settable dose. The dose may be set, preferably in quantities corresponding to whole-number multiples of one unit dosage increment.

In one embodiment, the separation, e.g. the axial separation, between the first position and the second position is determined by, for example equal to, a switching distance, e.g. a clutch release distance. The switching distance may be the distance by which the second member of the dose setting and drive mechanism has to be moved relative to the first member of the dose setting and drive mechanism in order to switch the dose setting and drive mechanism from a dose setting configuration of the mechanism to a dose delivery configuration of the mechanism. In the first position, the dose setting and drive mechanism may be in the dose setting configuration. In the second position, the dose setting and drive mechanism may be in the dose delivery configuration. In the dose setting configuration or the first position, for example, the members of the dose setting and drive mechanism may be rotationally locked as has been discussed further above. In the dose delivery configuration or the second position, relative rotation is allowed, e.g. the first member may rotate relative to the second member and the housing during dose delivery. During the dose delivery operation, the second member may be rotationally locked relative to the housing.

In one embodiment, the, e.g. axial, separation between the first position and the second position is greater than or equal to the distance by which the second member has to be moved, e.g. axially, relative to the first member in order to release the rotational lock. Specifically, during the movement of the user interface member from the first position to the second position, the rotational lock may be released by displacing the second member axially, e.g. distally, relative to the first member. The distance to release the rotational lock may correspond to the switching distance. As the activation operation may require a movement towards the first position, the switching of the electronic system to the second state may take place when the first and the second member are or are being rotationally locked to one another which is a stable state and, hence, can be reliably handled.

In one embodiment, in order for the manipulation to qualify as an activation operation, it may be required that the size of the currently set dose is zero. That is to say, the manipulation may only then be qualified as an activation operation when there is no dose set. In this way, it can be ensured that the activation operation needs to take place before the dose setting operation is commenced, even if the motion sensing unit and/or the communication unit only need to be operational for the dose delivery operation to deliver the dose set in the dose setting operation. This gives a safety margin for the system to switch from the first state into the second state and ensures that the electronic functionality is available timely, e.g. for the dose delivery operation.

In one embodiment, the electronic system comprises a power supply, e.g. a rechargeable or non-rechargeable battery. The resources of the power supply may last longer when the activation operation is performed before the system is switched to the second state than when the electronic functionality is available all the time. Also, if a manipulation different from the setting operation is required, playing around with the system with setting and unsetting doses repeatedly will not drain substantial power.

In one embodiment, the electrical signaling unit comprises a switch, e.g. an electrical switch. The switch may be configured to or triggered to provide a switch signal, e.g. when the user interface member moves from the first position to the second position and/or when the user interface member is touched. The switch signal may be caused by a manipulation of the user interface member, e.g. a movement of the user interface member. The switch signal may be or may comprise a change in an electrical signal or potential. The signal may decrease, e.g. cease or return to zero or to a non-significant signal, when the user interface member moves towards the first position, e.g. when starting from the second position. That is to say, the signal may decrease or vanish when the user interface member again assumes its first position relative to the housing. In the first position the switch may be open and no signal is generated. In the second position, the switch may be closed and the switch signal is generated.

In one embodiment, the switch signal has a signal portion characteristic for the movement from the second position to the first position. The manipulation evaluation unit may be configured to qualify a manipulation as an activation operation only when the manipulation evaluation unit recognizes the signal portion, preferably based alone on the signal portion.

The signal, e.g. a current or a voltage, may comprise a first portion and a second portion. The first portion of the signal may be an increasing flank or edge of the signal. The second portion of the signal may be a decreasing flank or edge of the signal. Alternatively, the first portion may be a decreasing flank or edge of the signal and/or the second portion may be an increasing flank or edge of the signal. The second portion may terminate the signal, the first portion may be the initial portion of the signal. An intermediate portion of the signal may directly connect the first and second portions. The intermediate portion may have a continuous, e.g. non-zero signal, which preferably is significantly distinguished from noise. The signal strength, e.g. the current or voltage, in the intermediate portion may be constant. The second portion may be a signal portion characteristic for opening a closed switch. The first portion may be characteristic for closing a switch. When the switch is closed, current may flow through the switch.

In one embodiment, the signal portion characteristic for the movement from the second position to the first position has to occur within a predetermined time after the onset of the signal, e.g. within 10 s, such that the manipulation evaluation unit qualifies the manipulation as an activation operation.

In one embodiment, the duration of the switch signal has to be greater than a minimum duration, e.g. greater than 0.1 s, such that the manipulation evaluation unit qualifies the manipulation as an activation operation. Alternatively or additionally, the duration of the switch signal has to be less than a maximum duration, e.g. less than 10 s, such that the manipulation evaluation unit qualifies the manipulation as an activation operation.

In one embodiment, the manipulation evaluation unit is configured to qualify a manipulation as an activation operation only if a movement sequence has been completed or a signal portion or signal sequence characteristic for the operation or generated, preferably within a predetermined time. Alternatively or additionally, the manipulation evaluation unit may qualify a manipulation as an activation operation only if a movement or signal portion characteristic for the activation operation, e.g. opening a closed switch, has been completed or generated.

In one embodiment, the manipulation evaluation unit is configured to qualify a manipulation as an activation operation only when the manipulation evaluation unit recognizes a predetermined number, e.g. two, successive switch signals, preferably within a predetermined time. The respective switch signal may be a signal pulse. Each pulse may involve closing and opening the switch. That is to say, the manipulation which qualifies as an activation operation may require successive movements for closing and opening the switch, e.g. two times movement of the user interface member from the first position to the second position and back, e.g. by pressing and releasing the user interface member. The predetermined time may be less than 5 s, e.g. 2 s. If the predetermined number is not reached or exceeded, preferably within the predetermined time, the manipulation may not be qualified as an activation operation.

In one embodiment, the at least one signal, e.g. a portion of the signal, the sequence of the signals and/or the time characteristic of the signal or the sequence, generated by the signaling unit which is indicative for the activation operation is unique for the activation operation.

In one embodiment, the manipulation evaluation unit monitors signals generated by the signaling unit. The signals generated by the signaling unit may be associated with movements of the user interface member.

In one embodiment, the manipulation evaluation unit has two different states, a low power state and a higher power state, where the power consumption is higher in the higher power state than in the low power state. A signal generated by the signaling unit, e.g. any signal generated by the unit when the evaluation unit is in the low power state, may be used to switch the manipulation evaluation unit from the low power state to the higher power state, e.g. from non-operational to operational. Consequently, the signaling unit may wake the manipulation evaluation unit.

In one embodiment, the electronic system comprises a feedback unit. The feedback unit may be configured to generate a feedback perceivable by the user. The feedback may enable the user to determine whether the system is in the first state or in the second state. Preferably, in the first state, there is no perceivable feedback provided and the feedback is indicative for the second state. The feedback may be a feedback signal, such as an optical signal, for example. The feedback signal may be provided by a light source, such as a light-emitting diode. The light source may operate in a pulsed or flashing manner for providing the feedback.

In one embodiment, the signaling unit and/or the manipulation evaluation unit is integrated into the user interface member. The manipulation evaluation unit may be integrated into the electronic control unit or at least operatively connected thereto.

In one embodiment, the electronic system comprises one user interface member, e.g. one integral member, for the dose setting operation and the dose delivery operation or two different user interface members, where one of these members is the user interface member for dose setting and the other one is the user interface member for dose delivery. The two different members are expediently movable relative to one another, e.g. to switch between a dose setting configuration and a dose delivery configuration. If one interface member is used for dose setting and dose delivery, this interface member may have the setting surface and the delivery surface, which, preferably, are not movable relative to one another, especially not for or during dose delivery and/or not for or during dose setting. If two different user-interface members are used, the setting surface and the delivery surface may be on different members and movable relative to one another for or during dose delivery and/or for or during dose setting.

In one embodiment, the electronic system comprises a timer unit. The timer unit may be configured to deactivate the motion sensing unit and/or other electrically powered units of the electronic system after a predetermined time period has elapsed and, preferably when in this time period no motion signal and/or no use signal is generated. The timer unit may trigger or cause the electronic system to be switched from the second state back to the first state. In other words, the electronic system may be configured to switch from the second state back to the first state, preferably when for a predetermined time no motion signal is generated and/or received by the electronic control unit.

In one embodiment, the user interface member is a dose setting and/or an injection button of or for the drug delivery device.

In one embodiment, the drug delivery device comprises a reservoir retainer for retaining a reservoir with drug, e.g. a cartridge, and/or the device comprises the reservoir with drug. The reservoir may comprise drug sufficient for a plurality of, preferably user-settable, doses to be delivered by the drug delivery device.

In one embodiment, the drug delivery device is a pen-type device.

In one embodiment, the electronic system is configured as a, preferably reusable, add-on for a drug delivery device unit. The system may be configured to be attached to the drug delivery device unit. That is to say, the electronic system may be configured to be used with a plurality of drug delivery device units. The respective drug delivery device unit may be a disposable drug delivery device unit and/or the respective drug delivery device unit may be fully operational for performing dose setting operations and dose delivery operations. The drug delivery device unit may comprise the reservoir.

In one embodiment, a kit for a drug delivery device comprises the drug delivery device unit and the electronic system. The system may be attachable to the device unit to form the drug delivery device. Features disclosed above and below for the drug delivery device, especially the ones that are not directly related to the electronic system, should also apply for the drug delivery device unit and vice versa.

In one embodiment, the method of preparing an electronic system for a drug delivery device or a drug delivery device comprising an electronic system for a dose delivery operation comprises: evaluating at least one electrical signal provided by the electronic system in response to a manipulation of a user interface member, wherein it is evaluated whether the at least one signal indicates an activation operation of the user interface member and, in the affirmative, switching the electronic system to a state of higher power consumption for the dose delivery operation, wherein the manipulation of the user interface member indicative for the activation operation is different from a manipulation of the user interface member required for a dose setting operation and/or from the manipulation required for a dose delivery operation. The computer program product may comprise the criteria which have to be met such that the manipulation qualifies as or indicates an activation operation. Once activated and in the second state, the electronic system may be configured to gather and/or communicate dose data during the delivery operation or after completion of the delivery operation.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end, where a needle unit is or is to be mounted to the device, for example.

In a particularly advantageous embodiment, an electronic system for a drug delivery device comprises:
at least one user interface member configured to be manipulated by a user for performing a dose setting operation to set a dose of drug to be delivered by the drug delivery device and/or for performing a dose delivery operation for delivering a set dose,
an electronic control unit configured to control operation of the electronic system, the electronic system having a first state and a second state, wherein the electronic system has an increased electrical power consumption in the second state as compared to the first state,
an electrical signaling unit configured to provide at least one electrical signal when the user interface member is manipulated, wherein the user interface member is further configured to be manipulated for an activation operation,
a manipulation evaluation unit, the manipulation evaluation unit being operatively connected to the electrical signaling unit and configured to evaluate the at least one electrical signal of the signaling unit in order to determine whether the manipulation of the user interface member indicated by the at least one signal of the signaling unit qualifies as an activation operation, wherein the manipulation of the user interface member for the activation operation is different from the manipulation of the user interface member for the dose setting operation and for the dose delivery operation, and wherein
the electronic control unit is configured to switch the electronic system into the second state of higher power consumption when the manipulation evaluation unit has confirmed that the manipulation qualifies as activation operation.

Accordingly, a distinguished user action as activation operation is required to activate the electronic system. This user action may be different from the dose setting operation and/or the dose delivery operation as has been discussed above already.

Features, which are disclosed in conjunction with different aspects and embodiments may be combined with one another even if such a combination is not explicitly discussed above or below. Further aspects, embodiments and advantages will become apparent from the following description of the exemplary embodiments in conjunction with the drawings.

### BRIEF DESCRIPTION OF THE FIGURES

FIG. 1 illustrates an embodiment of a drug delivery device.

FIG. 2 illustrates schematically an electronic system for a drug delivery device, e.g. the one in FIG. 1.

FIGS. 3A and 3B schematically illustrate embodiments of the electronic system.

FIGS. 4A through 4C schematically illustrate signals generated by the signaling unit.

### DETAILED DESCRIPTION

In the drawings identical features, features of the same kind or identically or similarly acting features may be provided with the same reference numerals in the drawings.

In the following, some concepts will be described with reference to an insulin injection device. The systems described herein may be implemented in this device or used as an add-on to the device. The present disclosure is however not limited to such an application and may equally well be used for or in injection devices that are configured to eject other medicaments or drug delivery devices in general, preferably pen-type devices and/or injection devices.

In the following, embodiments are provided in relation to injection devices, in particular to variable dose injection devices, which record and/or track data on doses delivered thereby. These data may include the size of the selected dose and/or the size of the actually delivered dose, the time and date of administration, the duration of the administration and the like. Features described herein may include power management techniques (e.g. to facilitate small batteries and/or to enable efficient power usage).

Certain embodiments in this document are illustrated with respect to an injection device where an injection button and grip (dose setting member or dose setter) are combined e.g. similar to Sanofi's ALLSTAR® device. The injection button may provide the user interface member for initiating and/or performing a dose delivery operation of the drug delivery device. The grip or knob may provide the user interface member for initiating and/or performing a dose setting operation. The devices may be of the dial extension type, i.e. their length increases during dose setting. Other injection devices with the same kinematical behaviour of the dial extension and button during dose setting and dose expelling operational mode are known as, for example, the Kwikpen® or Savvio® device marketed by Eli Lilly and the FlexPen®, or Novopen® device marketed by Novo Nordisk. An application of the general principles to these devices therefore appears straightforward and further explanations will be omitted. However, the general principles of the present disclosure are not limited to that kinematical behaviour. Certain other embodiments may be conceived for application to injection devices where there are separate injection button and grip components/dose setting members e.g. Sanofi's SoloSTAR®. Thus, the present disclosure also relates to systems with two separate user interface members, one for the dose setting operation and one for the dose delivery operation. In order to switch between a dose setting configuration of the device and a dose delivery configuration, the user interface member for dose delivery may be moved relative to the user interface member for dose setting. If one user interface member is provided, the user interface member may be moved distally relative to a housing. In the course of the respective movement, a clutch between two members of the dose setting and drive mechanism of the device changes its state, e.g. from engaged to released or vice versa. When the clutch, e.g. formed by sets of meshing teeth on the two members, is engaged, the two members may be rotationally locked to one another and when the clutch is disengaged or released, one of the members may be permitted to rotate relative to the other one of the two members. One of the members may be a drive member or drive sleeve which engages a piston rod of the dose setting and drive mechanism. The drive sleeve may be designed to rotate relative to the housing during dose setting and may be rotationally locked relative to the housing during dose delivery. The engagement between drive sleeve and piston rod may be a threaded engagement. Thus, as the drive sleeve cannot rotate during dose delivery, axial movement of the drive sleeve relative to the housing will cause the piston rod to rotate. This rotation may be converted into axial displacement of the piston rod during the delivery operation by a threaded coupling between piston rod and housing.

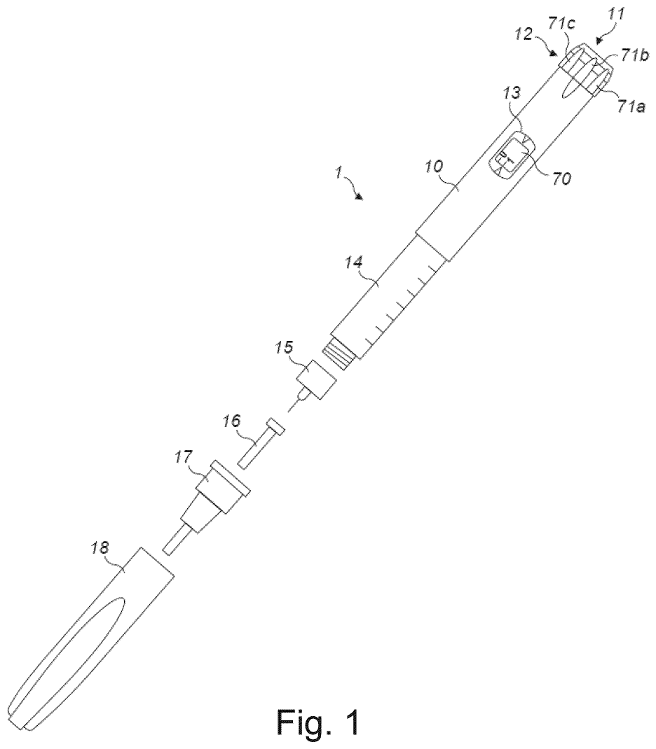

The injection device 1 of FIG. 1 is an injection pen that comprises a housing 10 and contains a container 14, e.g. an insulin container, or a receptacle for such a container. The container may contain a drug, e.g. insulin. The container may be a cartridge or a receptacle for a cartridge which may contain the cartridge or be configured to receive the cartridge. A needle 15 can be affixed to the container or the receptacle. The container may be a cartridge and the receptacle may be a cartridge holder. The needle is protected by an inner needle cap 16 and either an outer needle cap 17 or another cap 18. An insulin dose to be ejected from injection device 1 can be set, programmed, or ‘dialed in’ by turning a dosage knob 12, and a currently programmed or set dose is then displayed via dosage window 13, for instance in multiples of units. The units may be determined by the dose setting mechanism which may permit relative rotation of the knob 12 to the housing 10 only in whole-number multiples of one unit setting increment, which may define one dosage increment. This may be achieved by an appropriate ratchet system, for example. The indicia displayed in the window may be provided on a number sleeve or dial sleeve 70. For example, where the injection device 1 is configured to administer human insulin, the dosage may be displayed in so-called International Units (IU), wherein one IU is the biological equivalent of about 45.5 micrograms of pure crystalline insulin ( 1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10 or a transparent separate component inserted into an aperture of the housing, where the separate component may incorporate a magnifying lens. The dosage window 13 permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming.

In this example, the dosage knob 12 includes one or more formations 71a, 71b, 71c to facilitate attachment of a data collection device or electronic system. An electronic system which may be attachable to the user interface member (knob 12 and/or button 11) or, in general, to elements or members of a dose setting and drive mechanism of the drug delivery device 1 will be described in more detail below. The electronic system may be provided within the user interface member, for example. The electronic system which will be described in more detail below can also be configured as an add-on for a drug delivery device.

The injection device 1 may be configured so that turning the dosage knob 12 causes a mechanical click sound to provide acoustical feedback to a user. In this embodiment, the dosage knob or dose button 12 also acts as an injection button 11. When needle 15 is stuck into a skin portion of a patient, and then dosage knob 12/injection button 11 is pushed in an axial direction, the insulin dose displayed in display or dosage window 13 will be ejected from injection device 1. When the needle 15 of injection device 1 remains for a certain time in the skin portion after the dosage knob 12 is pushed home, the dose is injected into the patient's body. Ejection of the insulin dose may also cause a mechanical click sound, which is however different from the sounds produced when rotating the dosage knob 12 during dialing of the dose.

In this embodiment, during delivery of the insulin dose, the dosage knob 12 is returned to its initial position in an axial movement, without rotation, while the dial sleeve 70 or number sleeve 70 is rotated to return to its initial position, e.g. to display a dose of zero units. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g. 28 days after the first use) is reached.

Furthermore, before using injection device 1 for the first time, it may be necessary to perform a so-called “prime shot” to ensure fluid is flowing correctly from insulin container 14 and needle 15, for instance by selecting two units of insulin and pressing dosage knob 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

As explained above, the dosage knob 12 also functions as an injection button 11 so that the same component is used for dialling/setting the dose and dispensing/delivering the dose. Again, we note that a configuration with two different user interface members which, preferably only in a limited fashion, are movable relative to one another is also possible. The following discussion will, however, focus on a single user interface member which provides dose setting and dose delivery functionality. In other words, a setting surface of the member which is touched by the user for the dose setting operation and a dose delivery surface which is touched by the user for the dose delivery operation are immovably connected. Alternatively, they may be movable relative to one another, in case different user interface members are used. During the respective operation, the user interface member is preferably moved relative to the body or housing of the device. During dose setting the user interface member is moved proximally and/or rotates relative to the housing. During dose delivery, the user interface member moves axially, e.g. distally, preferably without rotating relative to the housing or body.

In the following, a general setup for an electronic system for a drug delivery device is disclosed.

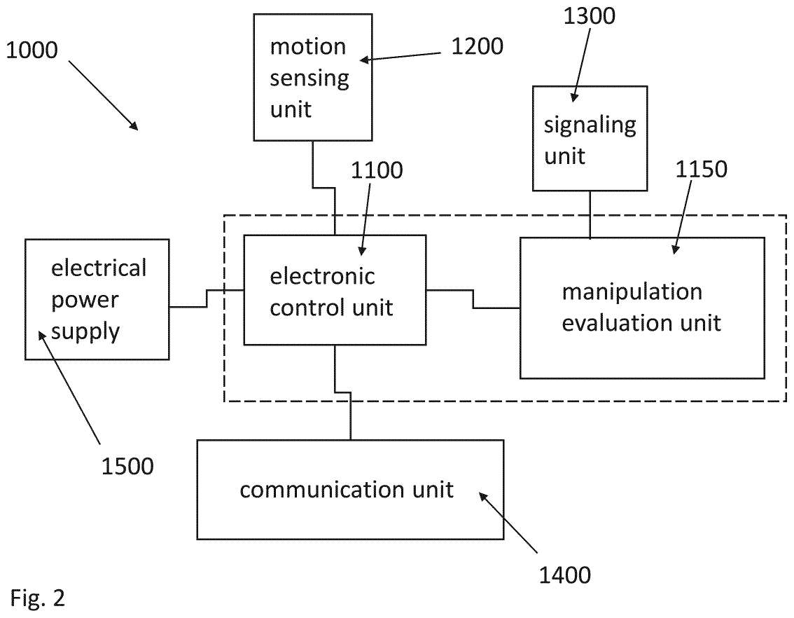

FIG. 2 illustrates a general configuration of elements of an electronic system 1000 which can be used in or for a drug delivery device, for example the device discussed further above or in other devices.

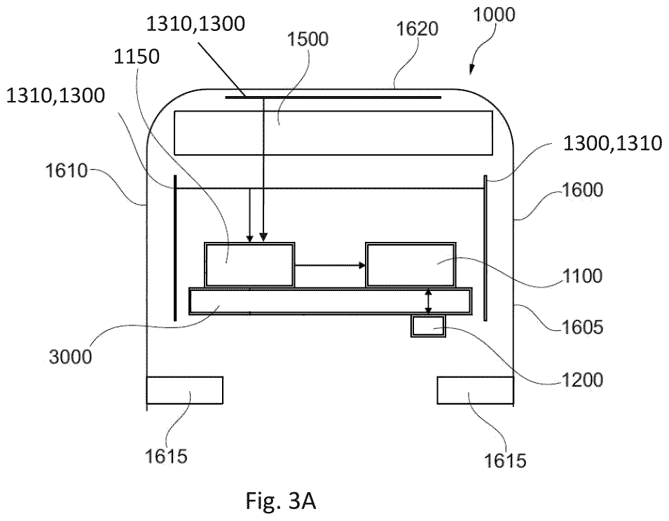

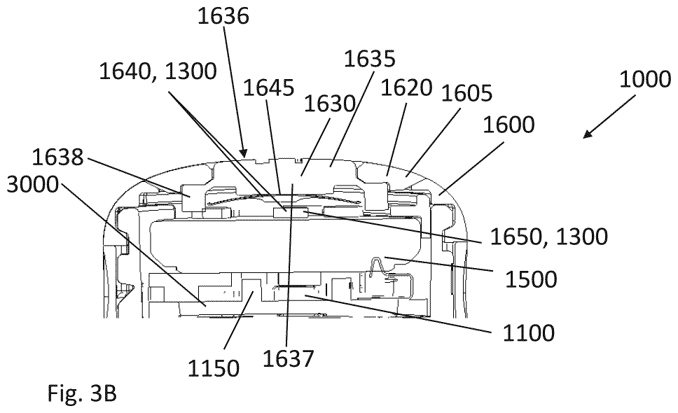

The electronic system 1000 comprises an electronic control unit 1100. The control unit may comprise a processor, e.g. a microcontroller or an ASIC. Also, the control unit 1100 may comprise one, or a plurality of memory units, such as a program memory and/or a main memory. The program memory may be designed to store program code which when carried out by the system controls operation of the system and/or the electronic control unit. The control unit 1100 is expediently designed to control operation of the electronic system 1000. The control unit 1100 may communicate via wired interfaces or wireless interfaces with further units of the electronic system 1000. The control unit 1100 may transmit signals containing commands and/or data to the respective unit and/or receive signals and/or data from the respective unit. The connections between the units and the electronic control unit 1100 are symbolized by the lines in FIG. 2. However, there also may be connections between the units, which are not illustrated explicitly. The control unit 1100 may be arranged on a conductor carrier, e.g. a (printed) circuit board (see reference 3000 in FIG. 3A). The other unit(s) of the electronic system may comprise one or more components which are arranged on the conductor carrier as well.

Electronic system 1000 further comprises an electrical motion sensing unit 1200. The motion sensing unit 1200 may comprise one sensor e.g. only one sensor, or a plurality of sensors. The motion sensing unit is expediently designed to generate motion signals, such as electrical signals, which are indicative for movement of one member of the electronic system or the drug delivery device relative to another member—e.g. movement of the dial sleeve or number sleeve relative to the drive sleeve or button/knob in the device discussed further above —, where the sensor may be fixedly connected to one of the members, e.g. the knob or button. The relative movement expediently occurs during the dose delivery operation. The respective sensor may be an optoelectronic sensor. The optoelectronic sensor may sense radiation emerging from a member moving relative to the sensor and impinging on the sensor to excite sensor signals or motion signals in the sensor, e.g. an optical encoder component. The radiation may be radiation reflected by the member and impinging on the member from a radiation source, such as an optoelectronic radiation source, e.g. an LED. The radiation source may be an IR source (IR-LED, an InfraRed Light Emitting Diode). The radiation source may be part of the sensor arrangement comprising the at least one sensor. One possible embodiment of the sensor is an IR-sensor which is configured to detect infrared light. The light source and the sensor may be arranged on the same component or member. The general functionality of optoelectronic sensor arrangements suitable for the electronic system discussed herein is disclosed in WO 2019/101962 A1, where the entire disclosure content is explicitly incorporated herein by reference for all purposes, especially as regards the different sensor arrangements and configurations. However, it should be noted that other sensor arrangement, e.g. using magnetic sensors, could be employed as well. In a motion sensing unit which has an electrically operated sensor and/or an electrically operated source for stimulating the sensor—such as a radiation emitter and an associated sensor—the power consumption may be particularly high and, hence, appropriate power management of electrical power available for powering the system may have a particular impact. The motion sensing unit 1200 may be designed to detect and preferably measure or quantify relative movement of one member of a dose setting and drive mechanism of or for the drug delivery device relative to another member of the dose setting and drive mechanism or relative to the housing 10 during a dose delivery operation. For example, the motion sensing unit may measure or detect relative rotational movement of two movable members of the dose setting and drive mechanism with respect to one another. Based on movement data received from or calculated from the signals of the unit 1200, the electronic system, e.g. the control unit, may calculate dose data, e.g. data on the currently delivered dose. The motion sensing unit 1200 is expediently configured to quantify the relative movement between a first member and a second member of the electronic system or the drug delivery device. The relative movement may be indicative for the delivered dose. The relative movement may be relative rotational movement. For example, the first member may rotate relative to the second member, such as during dose delivery. The motion sensing unit is expediently suitable to quantify the relative movement in whole-number multiples of one unit setting increment. The unit increment may be or may be defined by an angle greater than or equal to one of the following values: 5°, 10°. The unit setting increment may be or may be defined by an angle less than or equal to one of the following values: 25°, 20°. The unit setting increment may be between 5° and 25°, for example. The unit setting increment may correspond to a relative rotation of 15°, for example. The unit setting increment may be the rotation required to set the smallest settable dose to be delivered by the device. As has been explained above, the amount or distance of the relative (rotational) movement determined by the motion sensing unit between the first and second members is characteristic for the currently set dose in a dose setting operation or for the currently dispensed dose in a dose delivery operation. The size of the dose delivered may be determined by or correspond to the distance by which a piston rod of the dose setting and drive mechanism is displaced distally relative to the housing during the dose delivery operation.

The electronic system 1000 further comprises a signaling unit 1300. The signaling unit may be associated with the user interface member or members (knob 12 or button 11 in the device discussed above). Via the signaling unit 1300 the manipulation of the member for setting and/or for delivering a dose may be detected. The signaling unit is configured to generate an electrical signal or a sequence of electrical signals in response to a manipulation of the user interface member. The user interface member may have a setting surface which is arranged to be touched by the user for performing the dose setting operation and/or a delivery surface which is arranged to be touched by the user for performing the dose delivery operation. The setting surface may face in the radial direction and the delivery surface may face in the axial, e.g. proximal direction. Signal generation may require movement of the user interface member. Alternatively, proximity of the user to the surface of the user interface member, e.g. the setting surface and/or the delivery surface, or touching the user interface member may be sufficient for generating the signal. The element generating the signal may be an electrical sensor or switch, such as a micro force switch, for example. The signals generated by the signaling unit in response to a manipulation may allow to distinguish between different directions into which the user interface member is moved and/or different surfaces of the user interface member which are touched by the user or to which the user is close. The signaling unit is expediently configured such that the electrical signal(s) it is configured to generate responsive to a manipulation allow gathering information on what manipulation is currently being performed or has been performed, e.g. a dose setting operation, a dose delivery operation or a different operation. The signal(s) may allow to determine which surface of the user interface member is currently being touched or has(have) been touched during the manipulation, e.g. by the signal shape, occurrence and/or shape of characteristic portions of the signal or the sequence of the signal, e.g. the pattern, the frequency and/or their separation. Alternatively or additionally, the signal(s) may allow to determine a direction into which the user interface member has been moved, e.g. with respect to a housing such as housing 10 of the drug delivery device or the electronic system. The signaling unit may generate signals which are characteristic for one or more manipulations of the user interface member, e.g. on account of a sequence of signals or a portion of the signal. Examples for this will be discussed further below.

The electronic system 1000 further comprises a communication unit 1400, e.g. an RF, WiFi and/or Bluetooth unit. The communication unit may be provided as a communication interface between the system or the drug delivery device and an external device, such as other electronic devices, e.g. mobile phones, personal computers, laptops and so on. For example, dose data may be transmitted by the communication unit to the external device and/or synchronized with the device. The dose data may be used for a dose log or dose history established in the external device. The communication unit may be provided for wireless communication.

The electronic system further comprises a manipulation evaluation unit 1150. The manipulation evaluation unit is expediently operatively connected to the signaling unit 1300, e.g. wired or wireless. The manipulation evaluation unit 1150 may be configured to evaluate, e.g. on the basis of one or more signals received from the signaling unit 1300, the manipulation being performed or having been performed with the user interface member. The manipulation evaluation unit may monitor the signals generated by the signaling unit. Expediently, the manipulation evaluation unit is configured to determine, based on one or more signals received from the signaling unit, whether the user interface member is being manipulated in a manner which is characteristic for a dose setting operation, for a dose delivery operation and/or for another operation. For example, during a dose delivery operation a switch may be closed, e.g. for more than a predetermined time, e.g. more than 5 or more than 10 seconds, whereas in a dose setting operation, the switch may be open. The manipulation evaluation unit is configured to determine whether a manipulation which has been performed or is being performed with the user interface member qualifies or can be classified as an activation operation. Only if the manipulation qualifies as an activation operation, the manipulation evaluation unit issues an activation prompt or use signal which causes the electronic control unit 1100 to switch the electronic system from a first state or rest state (e.g. the state the system has when it is not needed, which is optimized in terms of power consumption) to a second state of higher power consumption, e.g. by activating the motion sensing unit 1200 and/or the communication unit 1400. For this purpose, the control unit 1100 may send an activation signal to the respective unit. In the second state, the motion sensing unit and/or the communication unit may be operable. In the first state, the motion sensing unit and/or the communication unit, preferably, cannot be operated. The manipulation evaluation unit 1150 is expediently operatively connected to the electronic control unit 1100 or integrated into that unit which is symbolized by the dashed box around the two units in FIG. 3A. The power consumption required in the first state for the signaling unit and the manipulation evaluation unit being operational advantageously is smaller than the power consumption when the communication unit and/or the motion sensing unit are operable. It is conceivable that an initial signal or the onset of the signal generated by the signaling unit acts as a wake up signal for the electronic control unit 1100 and/or for the manipulation evaluation unit such that the respective unit is woken from a state of even lower power consumption than it has in the state when the manipulation evaluation unit is operational. The activation operation of the user interface member is expediently different from the dose setting operation and from the dose delivery operation. In other words, the manipulation which has to be performed with the user interface member for the activation operation is expediently different from the setting operation and the delivery operation. Thus, a dedicated activation operation may be required to cause the system to switch to the second state. Hence, the user can decide whether the electronic functionality is required or, e.g. in view of urgency or other reasons, opt that the drug delivery device should be used without the electronic functionalities, which is preferably possible.

Criteria which the manipulation evaluation unit 1150 may use in order to evaluate whether the manipulation qualifies as an activation operation may comprise any one of, any arbitrarily selected plurality of or all of the following:
the user interface member is moved in two different, e.g. axial, directions such as relative to the housing 10 of the drug delivery device, of the drug delivery device unit or the electronic system. The different movements preferably have to occur close to one another in time, e.g. separated by less than 15 seconds, less than 10 seconds or less than 5 seconds. Performing a dose delivery operation for delivering a previously set dose usually takes longer than 5 seconds or 10 seconds. Hence, limiting the movements to a certain time interval, which preferably is less than the time interval typical for the duration of a delivery operation, reduces the risk of wrongly classifying a dose delivery operation as activation operation, when the user interface member is released after the delivery operation has been completed. Additionally or alternatively, requiring movements in different directions for the activation operation reduces the risk of accidentally performing an activation operation, e.g. by accidentally pushing on the user interface member with the content in a bag and retaining the user interface member in this state.

For example, an activation operation may require at least one movement of the user interface member in one direction and at least one movement of the user interface member in the opposite direction.

The number of movements in different directions which is required may be less than five sequential movements in different directions.
The time characteristic of signals or signal sequences generated by the signaling unit. For example, the separation between two successive signals and/or the duration of one signal or signal pulse may have to be less than or equal to a predetermined value, e.g. one of the following values: 10 s, 8 s, 6 s, 4 s, 2 s.
The occurrence of characteristic signal portions, sequences or patterns in the signals generated by the signaling unit.

For example, two signals, each of the signals being indicative for a movement of the user interface member in two different directions may have to occur within less than 5 s or 2 s.
The size of the set dose is zero. An electrical zero dose confirmation unit (not explicitly shown) may be provided in the system to confirm that the size of the currently set dose is zero or at least less than a minimum settable dose, particularly when the manipulation is evaluated by the manipulation evaluation unit in order to qualify the manipulation as an activation operation. The zero dose confirmation unit may comprise an electrical switch. The switch may be triggered to generate a switch signal when the user interface member is moved in the dose setting operation, e.g. rotated, away from its initial and/or zero dose position. Thus, the state of the switch prior to the rotation may be characteristic for a zero dose which is set. Absence of the switch signal prior to the manipulation which is currently evaluated—e.g. absence within a predetermined time interval before that manipulation such as 5 s or less—by the manipulation evaluation unit may be treated as confirmation that the size of the set dose was zero when the manipulation was initiated or is being performed. Alternatively, the zero dose confirmation unit may comprise an electrical dose size measuring unit, which is configured to measure the size of the currently set dose, e.g. by a rotational encoder. If the size of the set dose is greater than zero, the zero dose may be not confirmed by the zero dose confirmation unit. The zero dose confirmation unit may be operatively coupled to the electronic control unit, particularly such that the control unit may consider the state of the zero dose confirmation unit, i.e. whether the zero dose is confirmed or not. The activation operation may require that the set dose is zero.

These criteria may be implemented using comparatively simple switches or sensors in the electronic system 1000 where the signals are evaluated by the manipulation evaluation unit 1150.

It should be noted that different criteria or additional criteria may be applied as well. For example, a predetermined sequence of touch events occurring on different surfaces of the user interface member or tapping patterns occurring on one surface or combinations thereof may be used as criteria for the manipulation evaluation to qualify or classify that the manipulation is the activation operation.

As noted already, the activation operation is expediently distinct from the dose setting operation and the dose delivery operation. Specifically, the dose setting operation may require movement of the user interface member, e.g. rotation, in only one direction. The dose delivery operation may require movement of the user interface member also in only one direction, e.g. distally and only axially. Therefore, providing a manipulation evaluation unit which can distinguish another operation from the dose delivery operation and the dose setting operation, e.g. to trigger switching of the electronic system into a state with higher power consumption, is advantageous.

The electronic system 1000 further comprises an electrical power supply 1500, such as a rechargeable or non-rechargeable battery. The power supply 1500 may provide electrical power to the respective units of the electronic system.

In one embodiment, the power consumption, in particular the maximum power consumption, of the electronic system in the first state, e.g. prior to generation of the use or activation prompt signal, may be less than or equal to one of the following values: 300 nA, 250 nA, 200 nA (nA: nanoampere). Alternatively or additionally, in the second state of the electronic system, the power consumption, in particular the minimum power consumption, may be greater than or equal to one of the following values: 0.5 mA, 0.6 mA, 0.8 mA (mA: milliampere). The difference can result from the power consumption of the motion sensing unit 1200 and/or of the communication unit which may be active or operable in the second state and switched off or in a sleep state in the first state of the electronic system 1000.

In one embodiment, the power consumption P2 in the second state may be greater than or equal to at least one of the following values: 2*P1, 3*P1, 4*P1, 5*P1, 10*P1, 20*P1, 30*P1, 40*P1, 50*P1, 100*P1, 500*P1, 1000*P1, 2000*P1, 5000*P1, 10000*P1 where P1 is the power consumption in the first state. In the second state, the motion sensing unit may be active and/or the communication unit may be active, e.g. for wireless communication.

When the system is in the first state, e.g. with neither the motion sensing unit being active nor the communication unit, the current consumption may be 200 nA. When (only) the motion sensing unit is active, the power consumption may be 0.85 mA. When the communication unit is active, e.g. in addition to the motion sensing unit or only the communication unit, the power consumption may be 1.85 mA.

Although not explicitly depicted, the electronic system preferably comprises a, e.g. permanent and/or non-volatile, storage or memory unit, which may store data related to the operation of the drug delivery device such as dose (history) data, for example.

In one embodiment, the electronic control unit 1100 is configured to reduce the power consumption of the respective unit, i.e. to switch the unit back to the first state. This is suitable, for example, if an event which is relevant for that unit, e.g. a motion sensing event (motion signal) for the motion sensing unit, has not occurred in a predetermined time interval after the unit has been switched from the first state into the second state and/or after the use signal has been generated. The monitoring of the time interval may be achieved by a timer unit which is operatively connected to the electronic control unit (not explicitly shown). In case, after the use or activation prompt signal, there is no signal generated by the motion sensing unit within the predetermined time interval, the entire system may be switched to the first state again. This time interval may be greater than or equal to one of the following values 5 s, 10 s, 15 s, 20 s, 25 s, s. Alternatively or additionally the time interval may be less than or equal to one of the following values: 180 s, 150 s, 120 s, 90 s, 80 s, 70 s, 60 s, 50 s, 45 s, 40 s, 35 s, 30 s. The time interval may be between 5 and 180 seconds, e.g. 30 s or 180 s. The entire system may be switched back to the first state in case no motion signal is generated within the predetermined time interval. The predetermined time interval is expediently constant.

The respective unit which has been described above may be integrated into the user interface member of the electronic system which is discussed in further detail below in conjunction with various embodiments.

It goes without saying that the electronic system 1000 may comprise further electronic units other than the ones shown such as other sensing units, which sense or detect different quantities or events than the relative movements which the motion sensing unit detects.

In the following some more detailed embodiments of the electronic system are described. It should be noted that features which have been discussed above do also apply for these embodiments.

FIG. 3A schematically illustrates an embodiment of an electronic system 1000. The system 1000 comprises a user interface member 1600. The user interface member is designed to be operated during a dose setting operation and/or a dose delivery operation by the user. The user interface member 1600 has different exterior operation surfaces. The operation surfaces may be defined by exterior surfaces which are accessible from the exterior of a user interface member housing or body 1605. The user interface member 1600 has a setting surface 1610 which is arranged to be gripped by the user for dose setting, e.g. with two fingers such as the index finger and the thumb. The setting surface is a radially facing surface, which preferably circumferentially delimits the user interface member 1600 from the exterior. The user interface member 1600 also has a delivery surface 1620. The delivery surface is arranged to be contacted, e.g. pressed, by the user for dose delivery. The delivery surface 1620 is an axially oriented surface, e.g. a proximally facing surface. As noted above, embodiments of the disclosure can employ different user interface members for setting and delivery.

Within the user interface member 1600, e.g. within an interior hollow defined by the user interface member body 1605, some additional elements or units of the electronic system are housed. Specifically, the electronic system comprises the electronic control unit 1100. The system also comprises a conductor carrier 3000, e.g. a circuit board such as a printed circuit board. Conductors on the conductor carrier may conductively connect the electronic control unit to further electrical or electronic units or members of the system. The electronic control unit is arranged on the conductor carrier, e.g. mounted to the carrier.

The electronic system 1000 comprises a signaling unit 1300. In the depicted embodiment, the signaling unit has at least one sensor or switch or a plurality of sensors or switches 1310. In the depicted embodiment, at least one sensor or switch 1310 is associated with the setting surface 1610. Alternatively or additionally, at least one sensor or switch 1310 is associated with the delivery surface 1620. The respective sensor or switch is expediently configured to generate an electrical sensor signal or switch signal when the user interface member is moved or touched for being moved for performing the dose setting operation (the sensor or switch is expediently associated with the setting surface) or the dose delivery operation (the sensor or switch is expediently associated with the delivery surface). For example, for the dose setting operation, the user interface member 1600 may be rotated relative to the housing 10. For the dose delivery operation, the user interface member can be moved axially towards the housing, e.g. to switch the clutch such as from the state where the dial sleeve and the drive member are rotationally locked for dose setting to a state where relative rotation is allowed for dose delivery. The user interface member is preferably biased, e.g. by a clutch spring, to the position it has for dose setting which may be proximally offset to the one for dose delivery by the clutch switching distance. The clutch switching distance (the distance the user interface member has to be moved in order to switch the clutch) is for example greater than or equal to 1.5 mm.

The system 1000 further comprises the manipulation evaluation unit 1150, which receives electrical signals from the signaling unit 1300, e.g. from the respective sensor or switch thereof. As noted above, the manipulation evaluation unit 1150 may either communicate with the electronic control unit 1100 or be integrated therein. A signal sequence or signal generated during a manipulation of the user interface member can be evaluated in the evaluation unit. During evaluation it may be checked, whether the signal or the signal sequence meets one or more predefined criteria, e.g. the criteria mentioned above in conjunction with the description of FIG. 2.

The criteria are preferably linked to, e.g. unique for, the occurrence of the activation operation which the user should perform to activate one or more of the electronic functionalities of the system, such as the motion sensing unit 1200 and/or the communication unit 1400 which is not depicted in this embodiment but nevertheless preferably present. Only if the manipulation meets the criteria, the manipulation is classified as or qualifies as activation operation by the manipulation evaluation unit. Once the manipulation evaluation unit 1150 has determined that the activation operation has occurred, the evaluation unit 1150 issues the activation prompt signal or the use signal such that further electrical or electronic units of the system are activated, such as switched on, woken, or otherwise rendered operational, e.g. the motion sensing unit 1200 and/or the communication unit 1400, by the electronic control unit 1100. For activating the respective unit, the electronic control unit 1100 may issue an activation signal to the unit to be activated.

As already discussed, the manipulation required for the activation operation is expediently different from the dose setting operation (rotating the user interface member to increase the size of the set dose in one direction or to decease the size of an already set dose in the opposite direction) and the dose delivery operation move the user interface member axially towards the housing to drive a piston rod of the dose setting and drive mechanism.

The system furthermore comprises the motion sensing unit 1200 which is only schematically represented and, preferably, comprises one or more optoelectronic sensors and/one or more associated radiation emitters, e.g. IR sensors and IR emitters. The motion sensing unit may be bidirectionally conductively connected to the electronic control unit 1100 as hinted by the double arrow. One direction may be the one where the activation signal is transmitted from the electronic control unit to the motion sensing unit. In the other direction, motion signals may be sent from the motion sensing unit to the control unit, which may process the signals further, e.g. to calculate dose information or data. The motion sensing unit 1200 may be arranged on that side of the conductor carrier 3000 which faces away from the control unit 1100.

Further, the system 1000 comprises the power supply 1500, e.g. a battery, such as a coin cell. The power supply may be configured to provide a total charge of approx. 25-500 mAh at a voltage of approx. 1.4-3V. This may be achieved or assisted by stacking multiple coin cells, for example. The power supply 1500 is conductively connected or connectable to the other components of the electronic system, which require electrical power for operating. The conductive connection is not explicitly illustrated in FIG. 3A. The power supply may, however, be arranged so as to extend along one main surface of the conductor carrier 3000 as depicted. The power supply, in the depicted embodiment, is arranged between the conductor carrier 3000 and the delivery surface 1620. This facilitates a compact formation of the user interface member.

A radial width or diameter of the user interface member as seen from the exterior of the member, e.g. in top view onto the delivery surface, may be less than or equal to one of the following values: 2 cm, 1.5 cm. Alternatively or additionally, the radial width or diameter of the user interface member may be greater than or equal to one of the following values: 0.5 cm, 0.7 cm. The radial extension may be determined relative to the rotation axis of the user interface member during dose setting or relative to the main longitudinal axis of the user interface member, which axes may coincide. The length or axial extension of the user interface member 1600 may be less than or equal to one of the following values: 2.5 cm, 2 cm, 1.5 cm. Alternatively or additionally, the length or axial extension of the user interface member 1600 may be greater than or equal to one of the following values: 0.5 cm, 0.7 cm.

Electronic system 1000 is configured to be connected, preferably releasably, to a drug delivery device unit as an add-on unit or module. The drug delivery device unit may be electronic free. Accordingly, all electronics may be provided in the electronic system. The drug delivery device unit may be disposable. That is to say, the unit can be disposed of after a reservoir of the unit has been emptied using the drug delivery device comprising the unit and the system 1000. The electronic system 1000 could be reused for another drug delivery device unit. The drug delivery device unit is preferably configured as fully functional on its own, i.e. it could be operated for setting a dose to be delivered and deliver the set dose. One exemplary unit is the one depicted in FIG. 1. The electronic system may be a pure add-on to an, otherwise, fully functional unit. Alternatively, a drug delivery device may comprise the electronic system as an integral part, i.e. a part which is disposed of together with the remainder of the device and/or necessary such that the device can be operated for setting and delivering a dose of drug, e.g. because without the electronic system the drug delivery device unit would lack a surface accessible for the user for conducting a dose setting operation or a dose delivery operation. For a connection to the drug delivery device unit, the electronic system may comprise one or more connection features 1615, e.g. snap features. The respective connection feature is arranged in a distal portion of the user interface member 1600, e.g. in the interior of the member.

The system 1000 is expediently configured to be mechanically connected, either permanently or removably/releasably, to a member of the drug delivery device unit such as a member of the dose setting and drive mechanism, e.g. to a drive sleeve or the dose knob and/or the injection button of the unit discussed in conjunction with FIG. 1. The system, e.g. via the user interface member body 1605, may be rotationally and axially locked to the member of the drug delivery device unit. The member to which the system is connected may be movable relative to the housing 10 during dose setting and/or dose delivery, e.g. rotationally and/or axially during setting and, e.g. only, axially during delivery. The member can engage the piston rod, e.g. threadedly. The dose knob and the drive sleeve of the unit in FIG. 1 may be formed integral or act as a single member during dose setting and dose delivery. During dose setting, the drive sleeve may be selectively rotationally locked to a dial sleeve of the dose setting and drive mechanism such that the dial sleeve and the drive sleeve co-rotate during dose setting, e.g. by a clutch, and the dial sleeve rotates relative to the drive sleeve during dose delivery. The dial sleeve may be the number sleeve. The relative rotation between dial sleeve and drive sleeve during dose delivery may be measured by the motion sensing unit. However, it will be readily apparent to those skilled in the art that the disclosed concepts will also work with dose setting and drive mechanisms having different ways of operation and/or different configurations.

FIG. 3B illustrates another embodiment of an electronic system on the basis of a schematic sectional view. Again, as in the previous embodiment, this embodiment requires a separate user step in order to switch the electronic system into the second state with higher power consumption. It will be appreciated that features described for the preceding embodiments can also be applied for this embodiment and vice versa. Hence, the following description focusses on what has not been described already further above.

FIG. 3B illustrates the proximal portion of the user interface member 1600 of the electronic system 1000 with the delivery surface 1620. Accessible on the delivery surface 1620, the user interface member comprises a trigger section 1630. The trigger section 1630 is used to trigger the signaling unit 1300 to generate one or more signals in response to manipulations performed with the user interface member 1600 by the user. The signals can be evaluated in the manipulation evaluation unit 1150. The trigger section 1630 may be a surface section which is displaceable relative to the neighboring region of the delivery surface 1620, which may be formed by the user interface member body 1605. The trigger section 1630 may be formed by a member, e.g. a rigid member, which is received within an opening in the user interface member body 1605 and is accessible on the side of the delivery surface 1620. However, other configurations of the trigger section are also conceivable.

In case the trigger section 1630 is formed by a separate member from the user interface member body 1605, the member is expediently displaceable or movable relative to the user interface member body 1605, e.g. from an initial position such as depicted in FIG. 3B into the distal direction relative to the user interface member body 1605 until the member is sub-flush to the exterior surface of the user interface member body, i.e. recessed distally relative to that surface, in the end position. The member for the trigger section 1630 is therefore referred herein as movable member. Reference 1635 is used in the drawings for the movable member. In the initial position the movable member 1635 may have a proximally facing surface 1636 which defines the exterior contour of the user interface member 1600. The exterior contour is preferably smooth. That is to say, the trigger section 1630 in its initial position, preferably, does not protrude from the user interface member body 1605 but continues the proximal surface of that body in the region of the opening smoothly on the exterior. The opening may be a central opening in the user interface member body. Specifically, the longitudinal axis or the rotation axis for the dose setting operation may run through the opening and the movable member 1635. The member 1635 has a central or first portion 1637 received in the opening. One or more second portions 1638 protrude from the first portion e.g. radially outwardly. The second portion may be less rigid than the first portion. The second portion may be arranged below the proximal surface of the user interface member body 1605 adjacent to the opening. The member 1635 is expediently biased towards its initial position. That is to say, once the movable member has been displaced away from that position towards an end position, and the force causing that displacement is removed from the member a biasing force reestablishes the initial configuration depicted in FIG. 3B, i.e. moves the member into its initial position.

In the depicted embodiment, below the trigger section 1630, preferably immediately below and/or offset in the distal direction from the movable member 1635, a switch arrangement or switch 1640 is provided as the signaling unit 1300 in the present embodiment. In the initial position of the movable member 1635 depicted in FIG. 3B, the switch is expediently open. This reduces the power consumption when the switch is not triggered. When the movable member, which is touched by the user, is moved when pressing on the user interface member in the distal direction, the switch may be triggered or closed. In the depicted embodiment, the switch 1640 comprises a first switch feature 1645, which is expediently electrically conductive. The first switch feature 1645, e.g. a snap dome such as a metallic snap dome, may be arranged to mechanically contact a second switch feature 1650, e.g. electrically conductive. The mechanical contact triggers a signal, e.g. current flowing through the contact area between the switch features. The power may be provided by the power source 1500. While the switch is closed, the power consumption of the system may be increased, but preferably still be below the power consumption with the motion sensing unit and/or the communication unit being operable. The switch 1640 may be a single-pole single-throw switch. The movable member may be in mechanical contact with the first switch feature 1645 regardless of the position it has relative to the user interface member body 1605.

The switch is operatively connected to the manipulation evaluation unit 1150 and/or the electronic control unit 1100. Switch 1640 and electronic control unit 1100 and/or manipulation evaluation unit 1150 may be arranged on two different sides of carrier 3000 and/or power supply 1500. This facilitates a compact arrangement of the components or units within the user interface member body 1605. One or more components of the motion sensing unit such as light emitting diodes, optoelectronic sensors and/or light guides may be arranged below the carrier 3000 and/or the power supply 1500 (not explicitly highlighted in the drawings).

When the movable member 1635 for the trigger section 1630 is in the end position (distally offset from the position it has in FIG. 3B) where the switch 1640 is closed, it is biased towards the initial or proximal position. The biasing may be effected by the switch feature 1645, which may be elastically deformed from its original shape when the movable member 1635 is in the end position. The switch feature may be a snap dome. Alternatively, the movable member 1635 itself may be elastomeric and/or elastically deformable such that it, once deformed, assumes its original shape when the force causing the deformation is removed.

When the movable member is sub-flush relative to the user interface member body 1605, i.e. in the situation when the movable member has been moved from its initial position to the end position, the predominant part of the force exerted by the user onto the user interface member 1600 and particularly its delivery surface may be transferred through rigid parts of the electronic system, e.g. the user interface member body 1605 and/or parts connected thereto such as a chassis, e.g. a light guide chassis (light guides for the motion sensing unit may be part of or be carried by the light pipe chassis, not explicitly shown). A direct transmission of force required to operate the drive mechanism from the user interface member to the piston rod through the electronic components of the electronic system may therefore be avoided in the dose delivery operation.

In the present embodiment, when the switch 1640 is triggered or closed the electronic control unit 1100 does not activate the motion sensing unit. Rather, the system 1000 is configured to wait until the switch is opened again, before the electronic control unit 1100 issues a command or signal to switch the electronic system to the state of higher power consumption, e.g. by activating the motion sensing unit and/or the communication unit (not explicitly shown). This functionality can be implemented into the software governing operation of the electronic system and accordingly controlling operation of the manipulation evaluation unit. Hence, the signal provided by the switch can be evaluated in the evaluation unit 1150 (which may be integrated into the control unit 1100 or separate therefrom) until a portion of the signal is detected, which is characteristic for the transition of the switch (which is normally open) from closed to open. This portion may be a falling edge or flank of a current through the switch when the current, which is at its maximum when the switch is closed, decreases, e.g. below 50% of the maximum. Alternatively, the switch signal which is evaluated may be a voltage, such as a voltage at a port or channel of the electronic control unit which is operatively coupled, e.g. conductively connected to a portion of the switch, such as the switch feature 1645. The change in voltage during closing and/or opening of the switch may be used as the switch signal. Depending on the layout of the system, when the switch is opened again after it has been closed, the voltage may either decrease or increase in the characteristic portion of the switch signal which is characteristic for opening a previously closed switch. Hence, in this embodiment, the electronic system is preferably configured to enable the motion sensing unit to be initialised on the falling edge of the current through a normally open, e.g. single-pole single-throw, switch (i.e. when the switch feature 1645 moves from the end position to the initial position). The current through or the voltage at the switch, expediently the voltage at that side of the switch or at the switch feature which is connected to the electronic control unit, can be used as switch signal which is evaluated in the manipulation evaluation unit as discussed above. In this manner, the motion sensing unit may be enabled or activated during travel between a user interface member position commensurate with the clutch in the drug delivery device unit discussed further above, which, when engaged (which it is in the initial position of the user interface member), rotationally locks two members of the dose setting and drive mechanism. It will be appreciated that the proposed concept can be adapted for different switch or sensor layouts, switch types or constructions.

Moreover, the evaluation unit 1150 is preferably configured to qualify a manipulation of the user interface member 1604 indicating that the electronic system should be switched to the second state of higher power consumption only if the characteristic portion in the signal of the signaling unit is detected within a predetermined time from an onset of the signal. The predetermined time may be 10 s or less. Alternatively or additionally, the characteristic signal or signal portion may have to occur after a minimum time has elapsed from the onset of the signal, e.g. 0.1 s. Thus, pressing and releasing the user interface member or closing and opening the switch may have to occur within 0.1 to 10 s such that the manipulation qualifies as the activation operation which should switch the system into the state of higher power consumption.

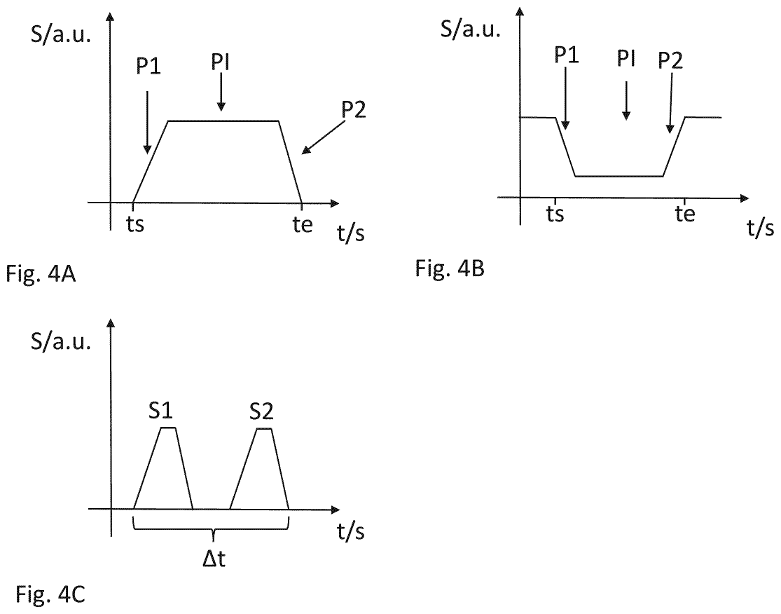

FIG. 4A illustrates schematically the signal S generated by the switch 1640 according to one embodiment. The switch is triggered or closed at time ts. As discussed further above already, the signal S may be the current through the switch or the voltage at the switch and/or the electronic control unit, e.g. at a port of the control unit which is associated with the switch. The signal increases in the first portion P1 of the signal up to the maximum in the intermediate portion PI. When the user releases the user interface member, the current decreases again in portion P2 until the signal terminates at time te. Portion P2 is the characteristic portion, which, once detected by the evaluation unit, causes the manipulation evaluation unit to qualify the manipulation of the user interface member as activation operation. The difference te−ts is preferably less than 10 s and greater than 0.1 s as has been discussed above. In order to qualify the portion P2 as the characteristic portion, the manipulation evaluation unit may have to detect a decrease in the current or voltage by 50% or more, e.g. from the maximum current or voltage, which may occur in the intermediate portion PI. In this embodiment, if the signal S is the voltage, the voltage initially is low and increases after triggering of the switch to a higher voltage. Thus, the port or channel of the electronic control unit connected to the switch may be at a low voltage, e.g. zero or non-zero, initially, i.e. before the switch is triggered or closed.

FIG. 4B illustrates another embodiment, where the signal S, e.g. the voltage at the switch or the electronic control unit, is initially high and is reduced when the switch is triggered or closed. In the intermediate portion PI, the signal may be zero or non-zero. The switch, when triggered may provide a connection to ground or 0V. This may cause the reduction in the voltage at the electronic control unit. Again, portion P2 which characterizes the switch returning into its initial condition, e.g. being open, is used as characteristic portion. Once this portion is detected, especially within the predetermined time interval after the switch has been triggered, the associated manipulation is qualified as an activation operation. The difference te−ts, again is preferably less than 10 s and greater than 0.1 s as has been discussed above. In order to qualify the portion P2 as the characteristic portion, the manipulation evaluation unit may have to detect an increase in the voltage by 50% or more, e.g. from the minimum voltage, which may occur in the intermediate portion PI. The signals in FIGS. 4A and 4B are somewhat complementary to each other.

Thus, the change from a low signal to a high signal (FIG. 4B) or from a high signal to a low signal (FIG. 4A), preferably with a previous change from a high signal to a low signal (FIG. 4B) or from a low signal to high signal (FIG. 4A), particularly within the predetermined time interval, may be used as characteristic portion or signal to qualify a manipulation as an activation operation.

Aside from using the characteristic portion of one (continuous) signal for indicating a manipulation which qualifies as activation operation it is also possible to use a sequence of separate succeeding signals or signal pulses, such as being generated when the user interface member of the trigger section is pressed and released repeatedly or tapped repeatedly, preferably within a predetermined time interval, such as twice within 2 s, as criterion for the activation operation. It will be appreciated that different criteria for manipulations which should be qualified as activation operation can be used. However, in the system, preferably only one manipulation—in addition to the dose setting operation and the dose delivery operation—is preferably chosen to qualify as activation operation.

FIG. 4C illustrates two successive (switch) signals S1 and S2 which occur due to repeated pressing and releasing of the user interface member 1600 within the time interval Δt, which may be 2 s. In the depicted embodiment, Δt covers the entire signal durations of both signals, i.e. from the onset or start of S1 to the end of S2. However, it is also possible to use two successive movements of the user interface member in the distal direction (i.e. pressing on the user interface member) as the manipulation which qualifies as activation operation. In this case, the time interval Δt may be between the onset of the signals S1 and S2, as it does not matter whether the user interface member is being released after the second time of pressing the user interface member, to qualify the manipulation as activation operation.

The manipulation evaluation unit 1150, preferably, is woken or rendered operational when the signaling unit generates a signal. Specifically, the signaling unit 1300 and/or switch 1640 may serve as a wake-up unit to activate the manipulation evaluation unit and/or the electronic control unit. The signalling unit may act as or be handled as an interrupt for the units it is designed to wake or activate. For example, the signaling unit may wake the electronic control unit, especially, if the manipulation evaluation unit is integrated into the control unit. The woken unit—e.g. the manipulation evaluation unit—may be kept operational until a predetermined time after the last signal has been generated, e.g. more than 10 s, has elapsed. Then the unit may be switched into a sleep or power off mode. Alternatively, the unit may be operational all the time. Once the occurrence of the activation operation has been affirmed by qualifying the manipulation as activation operation, the manipulation evaluation unit may be switched into a sleep or power off mode, preferably at least as long as the motion sensing unit and/or the communication unit is active. This may apply for all embodiments disclosed herein. In case the manipulation evaluation unit is integrated into the electronic control unit, it may be expedient to keep the manipulation evaluation unit operational, if the electronic control unit needs to perform functions at that stage. However, if the motion sensing unit performs measurements, the electronic control unit may be switched off for optimizing power consumption as its functionality may only be required after the measurement performed via the motion sensing unit has been completed. Generally, the electronic control unit may be set into a sleep or power off state, whenever its functionality is not required. The same holds for any other unit of the system, e.g. the communication unit.

Using manipulations to activate the electronic system which are different from the manipulations required for dose setting (rotating the dose setting member or user interface member) and dose delivery (moving the user interface member distally) is advantageous, as the user can decide whether the system should be operated in a higher power state. Integrating this functionality into one user interface member together with the dose setting and/or dose delivery functionality is also advantageous since a separate member is not required.

As the motion sensing unit, in this embodiment, is only rendered operational when the switch 1640 is opened again, situations, when the user interface member or the trigger section 1630 is continuously pressed, e.g. in a bag, do not dramatically increase power consumption, as the motion sensing unit will not be activated unless the switch is opened again. The power consumption of the switch, is of course less than the one of the motion sensing unit, even when the switch is closed.

It should be noted that, as an alternative to a movable member 1635 which is provided in addition to the user interface member body 1605 on the delivery surface 1620, an integral delivery surface which is locally deformable, preferably elastically deformable, could also be employed to achieve the same functionality. During the deformation, the switch can be closed and, when the delivery surface resumes its original shape, the electronic system can be powered up.

In a preferred embodiment, the electronic system comprises an indicator which indicates to the user whether the electronic system is in the second state (not explicitly shown). Consequently, the user may verify whether the electronic functionality has been activated. The indicator, for example, is an electrically driven indicator. Such an indicator may, for example, comprise a visible indicator, such as comprising one or more light sources, e.g. LEDs, or a tactile indicator, such as a vibration device. The indicator is preferably only active in the second state, particularly, if the indicator is an electrically driven indicator. The indicator may have unique indications on whether the motion sensing unit or the communication unit or any other electronic unit which needs to be powered up is active or not, e.g. by associating dedicated colors to the respective unit being active. For example, the LED may emit light, when the motion sensing unit and/or the communication unit is active in a pulsed mode of operation. If the motion sensing unit or the communication unit is inactive, the LED expediently does not emit light or light of a different color. However, if the LED is switched off while the motion sensing unit and/or the communication unit is not active or operable, this is of course advantageous in terms of power consumption. Thus, it is advantageous, if the higher power consumption of the indicator is synchronized with the higher power consumption of the motion sensing unit. There are various locations, where an indicator could be placed in the electronic system. For example, it could be placed below the movable member 1635 which in itself may be an elastomeric and/or translucent component. The duty cycle and ‘on-off’ pattern style of the indicator expediently is configured to minimize current consumption of the electronic system while clearly indicating to the user that the motion sensing unit and/or the communication unit is operational. An LED which is switched on to emit a signal every five seconds has been found suitable for indicating that the electronic functionalities of the system are available, for example.

Under normal operation, e.g. when the electronic system is fitted to a device unit, the user would perform a manipulation of the user interface member 1600 for the activation operation. For example, the user may depress the user interface member 1600, e.g. for less than 10 s, and release it or tap or move the interface member repeatedly, e.g. twice within 2 s. The evaluation unit 1150, based on the signals generated by the signaling unit 1300 in response to actions or movements occurring during the manipulation, confirms that the manipulation is the activation operation. This is indicative that the system should be switched to the state of higher power consumption. In response to the confirmation, the electronic control unit activates the motion sensing unit and, preferably, the communication unit. The unit(s) may be activated, preferably only, for a predetermined time t (e.g. greater than or equal to 10 s, e.g. 30 s or 180 s). The control unit expediently also activates the indicator to indicate that the system is operational to the user and dose data can be gather by the system. The indicator may comprises one or more pulsed LEDs, e.g. operated to generate a visible signal every 5 s when activated. The time t is preferably chosen sufficient to enable the user to set the desired dose and, advantageously, to initiate the dose delivery operation. To ensure that the motion sensing unit remains enabled for the duration of delivery of the drug, the time t may be reset or extended upon observation of subsequent switching events and/or upon observation of a state change in the motion sensing unit indicative for a dispensing operation having occurred or currently occurring, e.g. due to the motion signals generated by the motion sensing unit. If a delivery operation is not detected by the motion sensing unit after the time t has elapsed, the system is switched off again, e.g. the motion sensing unit and/or the communication unit are deactivated, as may the indicator. The indicator may be switched off prior to other components or units of the electronic system, e.g. prior to the motion sensing unit and/or the communication unit, e.g. by 5 s and/or by a time defined by the interval between two successive visible signals generated by the indicator. This has the advantage that a situation can be avoided where the user confirms via the indicator that the system is still active just prior to delivering a dose and, between this confirmation and starting the dose delivery operation, the system is deactivated and the user does not notice. If, after initiation of the dose delivery operation, the user interface member is released for a time greater than a preset release time, e.g. for longer than 0.1 second, dose data indicative for the dose which has been delivered so far may be written into a memory. The data is derivable and preferably derived from the motion signals provided by the motion sensing unit. The respective signals prompting the control unit to issue the according commands or signals, e.g. indicative for the user interface member being released by the user during the delivery operation, may be generated by the signaling unit 1300. When the system detects that the user interface member has been released by the user, the motion sensing unit and/or the communication unit preferably stay active for a post release time, e.g. for two more seconds, in order to ensure that in case the dose delivery operation has only been interrupted and will be continued soon the motion sensing unit is still available to generate signals to cover the remainder of the delivery operation. After the dose delivery operation has been completed and/or the motion sensing unit has been switched off again, dose data is written to the memory and an attempt is made to establish a communication channel with another device, e.g. a mobile phone or another computing device, via the communication unit. The dose data on the delivered dose may be communicated to that device by the communication unit, when the channel has been established successfully. Thereafter, the electronic system may be switched back to the first state, where the power consumption is advantageously small as the motion sensing unit, the communication unit and/or the indicator are not active in this state.

It will be appreciated that there are numerous configurations which could be used to implement what is disclosed herein. For example, it is also be possible to have a defined sequence of touching of different surfaces with an appropriate sensor or switch arrangement for the signaling unit within the predetermined time interval as an activation operation.

As has been discussed above, managing electrical power consumption or the resources of a power supply (e.g. a rechargeable or non-rechargeable battery) in drug delivery devices comprising electronic systems, such as the injection devices discussed further above, or systems for drug delivery devices is a problem which needs to be addressed, e.g. in order to optimize the use of the capacity of the power supply and/or in consideration of the sometimes considerable shelf time of a drug delivery device or an electronic system has before it reaches the user or patient. It needs to be ensured that the electronic system still functions properly for the duration of its intended use, even when storing the system for a longer period.

The present disclosures has presented various concepts which can be implemented in drug delivery devices or electronic systems thereof or therefore, e.g. for improving the power management in the devices. Some concepts rely on providing electrical power to certain units of the device only when needed or when it is very likely that the power will be needed. For example, the device which has been mentioned above and which is described in WO 2019/101962 A1, for example, energizes a motion sensing unit (the sensor system with IR-LEDs and IR-detectors) of the device only when the injection button (as user interface member) is being pressed for performing a dose delivery (injection) operation. The rotation of the encoder component or encoder ring can be used by the sensing unit to gather data on movements which are indicative for the dose which has been delivered during the delivery operation, after the motion sensing unit has been energized. From the measured movement data, it can be calculated, how much of the drug has actually been delivered. The amount of the actually delivered drug does not necessarily coincide with the dose which was previously set in a dose setting operation, e.g. when the user interrupts the delivery operation before it has been actually completed. Accordingly, it is advantageous to measure movements occurring during the dose delivery operation which are correlated to the amount of drug which has been delivered already, e.g. to get insight on the current status or the progress of the delivery operation. The determined delivered dose may be communicated, preferably wirelessly to an external or remote device, e.g. a hand-held device such as a smartphone, e.g. via the communication unit. In this way a dose log on the doses delivered by the user may be established, which may be accessed by the user easily.

The proposed concepts are suitable for a large variety of drug delivery devices comprising electronic systems or for electronic systems for such devices not only for the devices described further above. The device may be an injection device and/or a pen-type device. The device may be configured to receive or comprise a medicament container or cartridge. The container or cartridge may be filled with liquid drug to be delivered by the device. The device may be designed to deliver a plurality of doses of the drug. Consequently, the container or cartridge may comprise drug in amount sufficient for several doses to be delivered by the device. The device may be re-usable or disposable, where a re-usable device may be provided with a replacement medicament container or cartridge when the current container or cartridge is considered empty or needs to be replaced for different reasons. A disposable device may be a single use device which is disposed after the medicament container has been emptied. The device may be a device of a dial extension type, that is to say a device which increases in length during the dose setting operation, where the increase in length is proportional to the size of the set dose. During the associated dose delivery operation, the length of the device may be decreased again, e.g. until the device resumes its original length, i.e. the length it had before the dose setting operation has been commenced. Alternatively, the length of the device may be independent of the size of the set dose, e.g. constant or substantially constant during dose setting and/or dose delivery. The dose setting operation may involve a, preferably rotational, movement, of a dose setting member as user interface member, e.g. a knob, button or grip component (as discussed further above already). The dose delivery operation may involve a, preferably axial, movement of a dose delivery member as user interface member, e.g. a button such as the injection button discussed further above. As already discussed further above, the dose setting member and the dose delivery member may be formed by a single, e.g. unitary, component, where, preferably, different surfaces of the component are manipulated during the dose setting operation and the dose delivery operation or, alternatively, the dose setting member and the dose delivery member may be separate components/interface members or parts with relative movement being possible between these members, e.g. to switch the dose setting and drive mechanism between a dose setting configuration and a dose delivery configuration. There may be relative movement between these components either during dose setting or dose delivery or during both operations. During the dose setting operation a lateral or side surface, i.e. a radially facing surface, of the dose setting member may be gripped by the user, e.g. with the thumb and index finger. During the dose delivery operation an axially, e.g. proximally, facing surface of the dose delivery member may be touched by the user, e.g. with the thumb. During the dose delivery operation, an axial force may be transferred by the user to the dose delivery member in order to initiate and/or to continuously drive the dose delivery operation using a dose setting and drive mechanism of the device which, aside from the user interface member, may comprise further members, such as a drive member and a piston rod, for example. The drive member may engage the piston rod. The drive member may be a drive sleeve. In one embodiment, the dose delivery member may be a drive member which engages the piston rod threadedly. The device may be a device as disclosed, for example, in WO 2015/028439 A1 the disclosure of which is incorporated herein by reference in its entirety. In this device, the knob/button may be rotationally locked with the dial sleeve or number sleeve during dose setting via an according clutch being engaged. When delivering the dose, the clutch is released and the knob is rotationally locked relative to the housing. The dial sleeve may rotated relative to the housing during dose delivery.

The device may be a needle-based device, i.e. the drug may be delivered into the body via a needle piercing the skin, or may be needle-free. The device may be a device with a delivery assist, e.g. a spring-assisted or spring-driven device. In such devices, the dose delivery operation by the user is assisted or entirely driven by energy provided by an energy storage member such as a spring. The energy in the storage member may be increased during the dose setting operation by the user or the energy storage member may be provided with the entire energy required to empty the medicament container pre-stored in the member by the manufacturer. In the latter case, the user does not need to provide energy to increase the energy stored in the energy storage member such as during the dose setting operation.

We note that the description of the embodiments focuses on the motion sensing unit gathering data during the dose delivery operation. It is, however, also possible to gather data during dose setting.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

The scope of protection is not limited to the examples given herein above. Any invention disclosed herein is embodied in each novel characteristic and each combination of characteristics, which particularly includes every combination of any features which are stated in the claims, even if this feature or this combination of features is not explicitly stated in the claims or in the examples.

### REFERENCE NUMERALS

1 injection device, drug delivery device or device unit
10 housing
12 dosage knob
11 injection button
13 window
14 container
15 needle
16 inner needle cap
17 outer needle cap
18 cap
70 dial or number sleeve
71a-c formation
1000 electronic system
1100 electronic control unit
1150 manipulation evaluation unit
1200 motion sensing unit
1300 signaling unit
1310 switch
1400 communication unit
1500 electrical power supply
1600 user interface member
1605 user interface member body
1610 setting surface
1615 connection feature
1620 delivery surface
1630 trigger section
1635 movable member
1636 surface
1637 first portion
1638 second portion
1640 switch
1645 switch feature
1650 switch feature
3000 conductor carrier

1-16. (canceled)

17. An electronic system for a drug delivery device, the electronic system comprising:
at least one user interface member configured to be manipulated by a user for performing a dose setting operation to set a dose of drug to be delivered by the drug delivery device and/or for performing a dose delivery operation for delivering a set dose, and is further configured to be manipulated for an activation operation;
an electronic control unit configured to control operation of the electronic system, the electronic system having a first state and a second state, wherein the electronic system has an electrical power consumption in the second state that is higher than an electrical power consumption of the electronic system in the first state;
an electrical signaling unit configured to provide at least one electrical signal indicating that the user interface member is manipulated; and
a manipulation evaluation unit operatively connected to the electrical signaling unit and configured to evaluate the at least one electrical signal in order to determine whether a manipulation of the user interface member indicated by the at least one electrical signal of the electrical signaling unit qualifies as the activation operation, wherein a manipulation of the user interface member for the activation operation is different from a manipulation of the user interface member for the dose setting operation and for the dose delivery operation,
wherein the electronic control unit is configured to switch the electronic system into the second state that has the higher electrical power consumption in response to the manipulation evaluation unit confirming that the manipulation qualifies as the activation operation.

18. The electronic system of claim 17, wherein the manipulation that qualifies as the activation operation requires a sequence of different movements of the user interface member.

19. The electronic system of claim 18, wherein the manipulation that qualifies as the activation operation requires the sequence of different movements to be completed within a predetermined time.

20. The electronic system of claim 17, wherein the manipulation that qualifies as the activation operation requires movement of the user interface member in opposite axial directions.

21. The electronic system of claim 17, wherein the manipulation that qualifies as the activation operation requires a repetition of one particular sequence of movements of the user interface member.

22. The electronic system of claim 17, wherein the user interface member is movable from a first position to a second position relative to a housing, and wherein the manipulation which qualifies as the activation operation requires a movement of the user interface towards the first position.

23. The electronic system of claim 22, wherein a separation between the first position and the second position is determined by a switching distance by which a first member of a dose setting and drive mechanism has to be moved relative to a second member of the dose setting and drive mechanism in order to switch the dose setting and drive mechanism from a dose setting configuration of the dose setting and drive mechanism to a dose delivery configuration of the dose setting and drive mechanism.

24. The electronic system of claim 23, wherein, in the first position, the first member and the second member are rotationally locked to one another, and in the second position, the first member is rotatable relative to the second member.

25. The electronic system of claim 23, wherein the first position is an initial position of the user interface member relative to the housing before the dose setting operation and/or the dose delivery operation is commenced.

26. The electronic system of claim 23, wherein the separation between the first position and the second position is equal to the switching distance.

27. The electronic system of claim 22, wherein the electrical signaling unit comprises an electrical switch configured to be triggered to generate a switch signal, as part of the at least one electrical signal, when the user interface member moves from the first position to the second position, and
wherein the switch signal decreases when the user interface member moves towards the first position from the second position.

28. The electronic system of claim 27, wherein the switch signal has a signal portion that indicates a movement of the user interface member from the second position to the first position, wherein the manipulation evaluation unit is configured to qualify the manipulation as the activation operation only when the manipulation evaluation unit recognizes the signal portion.

29. The electronic system of claim 28, wherein the manipulation evaluation unit is configured to qualify the manipulation as the activation solely by recognizing the signal portion.

30. The electric system of claim 28, wherein the signal portion indicates opening the switch.

31. The electronic system of claim 27, wherein the manipulation evaluation unit is configured to qualify the manipulation as the activation operation only when the manipulation evaluation unit recognizes a predetermined number of multiple successive switch signals within a predetermined time.

32. The electronic system of claim 17, further comprising:
a motion sensing unit; and/or
a communication unit,
wherein the motion sensing unit is configured to generate electrical motion signals suitable to quantify a relative movement of a first member relative to a second member, and
wherein the motion sensing unit and/or the communication unit is not operational in the first state and is operational in the second state.

33. The electronic system of claim 17, wherein the electronic system comprises a feedback unit configured to generate a feedback indicating whether the electronic system is in the second state, wherein the feedback is perceivable by the user of the electronic system.

34. The electronic system of claim 17, wherein the electronic system is configured as an add-on module for the drug delivery device.

35. A drug delivery device comprising:
a reservoir including a drug; and
an electronic system comprising
at least one user interface member configured to be manipulated by a user for performing a dose setting operation to set a dose of drug to be delivered by the drug delivery device and/or for performing a dose delivery operation for delivering a set dose, and is further configured to be manipulated for an activation operation,
an electronic control unit configured to control operation of the electronic system, the electronic system having a first state and a second state, wherein the electronic system has a high electrical power consumption in the second state that is more than a power consumption of the electronic system in the first state,
an electrical signaling unit configured to provide at least one electrical signal indicating that the user interface member is manipulated, and
a manipulation evaluation unit operatively connected to the electrical signaling unit, and configured to evaluate the at least one electrical signal in order to determine whether a manipulation of the user interface member indicated by the at least one electrical signal of the electrical signaling unit qualifies as the activation operation, wherein a manipulation of the user interface member for the activation operation is different from a manipulation of the user interface member for the dose setting operation and for the dose delivery operation,
wherein the electronic control unit is configured to switch the electronic system into the second state that has the high electrical power consumption in response to the manipulation evaluation unit confirming that the manipulation qualifies as the activation operation.

36. A method of preparing an electronic system for a dose delivery operation, the method comprising:
evaluating at least one electrical signal generated by the electronic system to determine whether the at least one electrical signal indicates an activation operation of a user interface member of the electronic system, wherein the at least one electrical signal is generated in response to a manipulation the user interface member, and wherein the at least one electrical signal generated in response a manipulation of the user interface member indicative for the activation operation is different from an electrical signal generated in response to a manipulation of the user interface member required for a dose setting operation and from an electrical signal generated in response to a manipulation required for the dose delivery operation; and
in response to determining that the at least one electrical signal indicates the activation operation, switching the electronic system from a state of low power consumption to a state of high power consumption for the dose delivery operation, wherein the electronic system consumes more power in the state of high power consumption than in the state of low power consumption.
